(19) United States
(12) Patent Application Publication
(10) Pub. No.: US 2018/0170823 A1
(43) Pub. Date: Jun. 21, 2018

(54) EFFICIENT PROCESS FOR CONVERTING METHANOL TO GASOLINE

(71) Applicant: ExxonMobil Research and Engineering Company, Annandale, NJ (US)

(72) Inventors: Suriyanarayanan Rajagopalan, Spring, TX (US); Mohsen N. Harandi, New Hope, PA (US); Karlton J. Hickey, Boothwyn, PA (US)

(21) Appl. No.: 15/825,224

(22) Filed: Nov. 29, 2017

Related U.S. Application Data
(60) Provisional application No. 62/434,448, filed on Dec. 15, 2016.

(51) Int. Cl. C07C 1/20; C07C 31/04; C07C 43/04; B01J 38/22; B01J 29/40

(52) U.S. Cl. CPC C07C 1/20; C07C 31/04; C07C 43/043; C10G 2400/30; B01J 29/40; C10G 2400/02; B01J 38/22

(57) ABSTRACT

Systems and methods are provided for the conversion of methanol to gasoline. Such methods may be performed be the sequential conversion of methanol to dimethyl ether, the conversion of dimethyl ether (and unconverted methanol, if present) to an intermediate olefin-rich product, and the oligomerization of the olefin-rich product to gasoline boiling range hydrocarbons.

### CROSS-REFERENCE TO RELATED APPLICATIONS

This application claims priority to U.S. Provisional Application Ser. No. 62/434,448 filed Dec. 15, 2016, which is herein incorporated by reference in its entirety.

### FIELD

This application relates to the field of upgrading methanol to gasoline.

### BACKGROUND

Various methods are known for converting methanol to gasoline. In a common example, methanol is converted to dimethyl ether in a first reactor and the the dimethyl ether is fed to a second, packed-bed reactor containing a high activity ZSM-5 catalyst and operating at a relatively high inlet temperature (e.g., 570-635° F.) in order to convert the dimethyl ether into a product stream that includes hydrocarbons boiling in the gasoline boiling range. Byproducts, such as ethane, propane, and butane are separated from the product stream before the product stream is fractionated into a light gasoline stream and heavy gasoline stream. The heavy gasoline stream is typically treated to remove durene before it is blended into fuel products. It would be desirable to provide other more efficient pathways to convert methanol to gasoline.

### SUMMARY

In an aspect, a method is provided for converting methanol to gasoline. The method includes converting methanol to an intermediate stream comprising dimethyl ether; exposing the dimethyl ether to a first catalyst in a reactor under first effective conversion conditions to produce an olefin-rich intermediate product, the first effective conversion conditions including an outlet temperature of less than 750° F.; and exposing the olefin-rich intermediate product to a conversion catalyst under second effective conversion conditions to form an oligomerized olefin effluent comprising C5+ olefinic compounds, wherein the second effective conversion conditions comprise a pressure of less than about 500 psig and a temperature of at least about 700° F. (371° C.).

### DRAWINGS

FIG. 1 is a schematic illustrating an exemplary process of converting methanol to gasoline according to one or more embodiments of the present invention.

### DETAILED DESCRIPTION

Systems and methods are provided for the conversion of methanol to gasoline. Such methods may be performed be the sequential conversion of methanol to dimethyl ether, the conversion of dimethyl ether (and unconverted methanol, if present) to an intermediate olefin-rich product, and the oligomerization of the olefin-rich product to gasoline boiling range hydrocarbons. Advantageously, such systems and methods may be performed to reduce the quantity of durene produced in the process, provide for consistent quality in gasoline product, and allow for the operation of lower operating temperatures for slowing catalyst deactivation.

Systems and methods disclosed herein provide for the conversion of methanol to gasoline by (a) converting methanol to an intermediate stream comprising dimethyl ether; (b) exposing the dimethyl ether to a first catalyst in a reactor under first effective conversion conditions to produce an olefin-rich intermediate product, the first effective conversion conditions including an outlet temperature of less than 750° F.; and (c) exposing the olefin-rich intermediate product to a conversion catalyst under second effective conversion conditions to form an oligomerized olefin effluent comprising C5+ olefinic compounds, wherein the second effective conversion conditions comprise a pressure of less than about 500 psig and a temperature of at least about 700° F.

The olefin-rich intermediate product formed by step (b) may comprise at least 50 wt % olefins and may comprise at least 15 wt % C1-C4 hydrocarbons. The olefin-rich intermediate product may also comprise less than 30 wt % C5+ aromatics, such as less than 25 wt % C5+ aromatics. In addition, the olefin-rich intermediate product may also comprise less than 7 wt % C10+ aromatics, such as less than 5 wt % C10+ aromatics.

Step (b) may also be performed with first effective conditions that include an inlet temperature of less than 650° F. The first catalyst employed in converting dimethyl ether to the olefin-rich intermediate product in step (b) may be a ZSM-5 zeolite. Similarly, the catalyst used to convert the olefin-rich intermediate product to an oligomerized olefin effluent in step (c) may also be a ZSM-5 zeolite. Advantageously, steps (b) and (c) may be performed without intermediate separation of the olefin-rich product produced in step (b). In some embodiments, steps (a) and (b) are performed in a first fluid bed reactor. Step (c) may optionally be performed in the step reactor as steps (a) and (b) or may be performed in a separate reactor.

As used herein, and unless specified otherwise, “gasoline” or “gasoline boiling range hydrocarbons” refers to a composition containing at least predominantly C5-C12 hydrocarbons. In one embodiment, gasoline or gasoline boiling range components is further defined to refer to a composition containing at least predominantly C5-C12 hydrocarbons and further having a boiling range of from about 100° F. to about 450° F. In an alternative embodiment, gasoline or gasoline boiling range components is defined to refer to a composition containing at least predominantly C5-C12 hydrocarbons, having a boiling range of from about 100° F. to about 450° F., and further defined to meet ASTM standard D439.

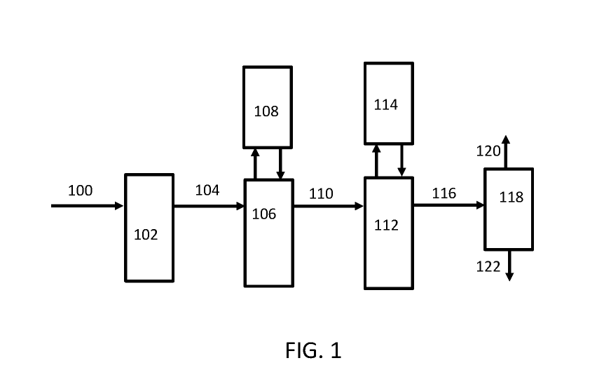

An exemplary embodiment is illustrated in FIG. 1. Systems and methods may include a feed stream 100 comprising methanol that is fed to a DME reactor 102 where at least a portion of the methanol in the feed stream 100 is converted to DME, such as through dehydration by exposing the methanol to a methanol dehydration catalyst.

The product stream 104, which includes DME and optionally methanol and/or water is fed to a reactor 106 where DME and methanol, if present, is converted to an olefin-rich product stream 110. Although not illustrated, at least a portion of methanol and/or water remaining in the product stream 104 may be separated from the product stream 104 prior to the product stream 104 being fed to reactor 106. Reactor 106 may be a fixed bed MTG catalytic reactor operating under conditions suitable for converting DME and methanol to an olefin-rich product, including C4 olefins, such as C2-C4 olefins. The olefin-rich product stream 110 may further include alkanes, such as C1-C5 alkanes. Regenerator 108 receives, periodically or continuously, catalyst from reactor 106, regenerates the catalyst, and feeds the catalyst back to reactor 106.

The olefin-rich product stream 110 is then fed to an oligomerization reactor 112, optionally without the intermediate separation of hydrocarbons present olefin-rich product stream 110. In oligomerization reactor 112, the olefin-rich product stream 110 may be exposed to a catalyst, under effective oligomerization conditions to convert the olefin-rich product stream 110 to as stream containing gasoline boiling range hydrocarbons 116. Reactor 112 may also include a regenerator 114 for regeneration of catalyst in reactor 112. The regenerator 114, similar to regenerator 108, may receive, periodically or continuously, catalyst from reactor 112 for regeneration.

The stream containing gasoline boiling range hydrocarbons 116 may then be fed to a separator 118, such as a debutanizer, to separate hydrocarbons boiling below the gasoline boiling range 120, such as C4 hydrocarbons, from the gasoline product 122.

### Converting Methanol to Dimethyl Ether

Various methods may be employed to convert methanol to dimethyl ether (“DME”). In any embodiment, methanol may be converted to DME with the use of a methanol dehydration catalyst. The methanol dehydration may be an acid catalyst, such as a zeolite, ion exchanged zeolite, a silicoaluminaphosphate (SAPO), alumina, alumina silicates, titania, zirconia, and mixtures thereof, or combinations of the acidic components thereof, e.g., WO3/ZrO2, ZrO2/SiO2, resins, MOFS, ZIFs. The acidic property can be Lewis acidity, or Bronsted acidity, and the combination of the both Lewis acidity and Bronsted acidity. The metal components can be mixed with acidic components, or impregnated onto acidic supports, or extruded with acidic components.

In any embodiment, a portion of the methanol in the feed may be converted to dimethyl ether leaving a portion of the resulting product stream as being unconverted methanol after exposure to methanol dehydration catalyst.

### Conversion of Dimethyl Ether to Olefin Product

Dimethyl Ether produced from the dehydration of methanol may be converted to an olefin product used a fixed be methanol-to-gasoline (MTG) catalytic reactor. The feedstock for the fixed bed MTG catalytic reactor can be the effluent from a DME dehydration reactor. In such a case, the effluent may be an equilibrium mixture of methanol, water and DME. If desired, complete or partial separation into constituent product streams of water, methanol, and DME may be carried out as described in U.S. Pat. No. 4,035,430 by condensation or fractionation, depending upon the degree of purity desired.

Developments in zeolite technology have provided a group of medium pore siliceous materials having similar pore geometry. Most prominent among these intermediate pore size zeolites is ZSM-5, which is usually synthesized with Bronsted acid active sites by incorporating a tetrahedrally coordinated metal, such as Al, Ga, or Fe, within the zeolytic framework. These medium pore zeolites are favored for acid catalysis; however, the advantages of ZSM-5 structures may be utilized by employing highly siliceous materials or crystalline metallosilicate having one or more tetrahedral species having varying degrees of acidity. ZSM-5 crystalline structure is readily recognized by its X-ray diffraction pattern, which is described in U.S. Pat. No. 3,702,866 (Argauer, et al.), incorporated by reference.

The preferred class of catalysts is characterized by a Constraint Index of 1 to 12 and a silica:alumina ratio of at least 12:1 and preferably higher e.g., 20:1 to 70:1, or even higher. Constraint Index of a zeolite is a convenient measure of the extent to which a zeolite provides constrained access to its internal structure for molecules of different sizes. It is therefore, a characteristic of the structure of the zeolite but is measured by a test which relies upon the possession of cracking activity by the zeolite. The sample of zeolite selected for determination of the Constraint Index of a zeolite should therefore represent the structure of the zeolite (manifested by its X-ray diffraction pattern) and have adequate cracking activity for the Index to be determined. If the cracking activity of the selected zeolite is too low, the Constraint Index may be determined by using a zeolite sample of the same structure but higher cracking activity which may be obtained, for example, by using an aluminosilicate zeolite of higher aluminum content. Details of the method of determining Constraint Index and of the values of the Index for typical zeolies are given in U.S. Pat. No. 4,016,218 to which reference is made for such details and other information in this respect.

Preferred zeolites which have the specified values of Constraint Index and silica:alumina ratio include zeolites having a ZSM-5 structure such as ZSM-5, ZSM-11, ZSM-12, ZSM-35, ZSM-38 and ZSM-48, which are described in U.S. Pat. Nos. 3,702,886 (ZSM-5), 3,709,979 (ZSM-11), 3,832,449 (ZSM-12), 4,076,842 (ZSM-23) and 4,016,245 (ZSM-35), 4,046,859 (ZSM-38) and 4,397,827 (ZSM-48), and reference is made to these patents for details of these zeolites, their preparation and properties. Of these zeolites, ZSM-5 is preferred.

The zeolite catalyst used is at least partly in the hydrogen form e.g., HZSM-5; but other cations, e.g., Periodic Groups III-VIII or rare earth cations may also be present. When the zeolites are prepared in the presence of organic cations they may be quite inactive possibly because the intracrystalline free space is occupied by the organic cations from the forming solution. The zeolite may be activated by heating in an inert atmosphere to remove the organic cations e.g., by heating at over 500° C. for 1 hour or more. The hydrogen form can then be obtained by base exchange with ammonium salts followed by calcination e.g. at 500° C. in air. Other cations e.g., metal cations can be introduced by conventional base exchange techniques.

Advantageously, the present methods and system allow for operation of the MTG reactor at lower temperatures than in traditional MTG operating schemes at least in part because alkanes and olefins boiling below the gasoline boiling range may be further converted to gasoline boiling range hydrocarbons in a downstream oligomerization reactor. Thus, the MTG reactor in the present systems and methods may be operated under effective conditions for producing an olefin-rich product, including an inlet temperature of less than 650° F. and an outlet temperature less than 750° F., or more preferably less than 750° F.

The hydrocarbon part of olefin-rich intermediate product may comprise at least 50 wt % olefins, and at least 15 wt % C1-C4 hydrocarbons. Furthermore, the olefin-rich intermediate hydrocarbon product may comprise less than 30 wt % C5+ aromatics, such as less than 25 wt % C5+ aromatics, or less than 20 wt % C5+ aromatics. For example, the olefin-rich intermediate product may comprise between about 1 wt % and 30 wt % C5+ aromatics, or between about 1 wt % and 25 wt % C5+ aromatics, or between about 1 wt % and 20 wt % C5+ aromatics. Furthermore, the olefin-rich intermediate hydrocarbon product may comprise less than 10 wt % C10+ aromatics, such as less than 7 wt % C10+ aromatics, or less than 5 wt % C10+ aromatics. For example, the olefin-rich intermediate product may comprise between about 1 wt % and 10 wt % C10+ aromatics, or between about 1 wt % and 7 wt % C10+ aromatics, or between about 1 wt % and 5 wt % C10+aromatics.

### Oligomerization of Olefin Product to Gasoline

The olefin product converted from dimethyl ether and optionally methanol may then be further converted to gasoline boiling range hydrocarbons by oligomerization. In various aspects, the olefin-containing product can be exposed to an acidic catalyst (such as a zeolite) under effective conversion conditions for olefinic oligomerization and/or sulfur removal. Optionally, the zeolite or other acidic catalyst can also include a hydrogenation functionality, such as a Group VIII metal or other suitable metal that can activate hydrogenation/dehydrogenation reactions. The olefin-containing product can be exposed to the acidic catalyst without providing substantial additional hydrogen to the reaction environment. Added hydrogen refers to hydrogen introduced as an input flow to the process, as opposed to any hydrogen that might be generated in-situ during processing. Exposing the olefin containing product to an acidic catalyst without providing substantial added hydrogen is defined herein as exposing a feed of the olefin-containing product to the catalyst in the presence of a) less than about 100 SCF/bbl of added hydrogen, or less than about 50 SCF/bbl; b) a partial pressure of less than about 50 psig (350 kPag), or less than about 15 psig (100 kPag) of hydrogen; or c) a combination thereof.

The acidic catalyst used in the processes described herein can be a zeolite-based catalyst, that is, it can comprise an acidic zeolite in combination with a binder or matrix material such as alumina, silica, or silica-alumina, and optionally further in combination with a hydrogenation metal. More generally, the acidic catalyst can correspond to a molecular sieve (such as a zeolite) in combination with a binder, and optionally a hydrogenation metal. Molecular sieves for use in the catalysts can be medium pore size zeolites, such as those having the framework structure of ZSM-5, ZSM-11, ZSM-12, ZSM-22, ZSM-23, ZSM-35, ZSM-48, or MCM-22. Such molecular sieves can have a 10-member ring as the largest ring size in the framework structure. The medium pore size zeolites are a well-recognized class of zeolites and can be characterized as having a Constraint Index of 1 to 12. Constraint Index is determined as described in U.S. Pat. No. 4,016,218 incorporated herein by reference. Catalysts of this type are described in U.S. Pat. Nos. 4,827,069 and 4,992,067 which are incorporated herein by reference and to which reference is made for further details of such catalysts, zeolites and binder or matrix materials.

Additionally or alternately, catalysts based on large pore size framework structures (12-member rings) such as the synthetic faujasites, especially zeolite Y, such as in the form of zeolite USY. Zeolite beta may also be used as the zeolite component. Other materials of acidic functionality which may be used in the catalyst include the materials identified as MCM-36 and MCM-49. Still other materials can include other types of molecular sieves having suitable framework structures, such as silicoaluminophosphates (SAPOs), aluminosilicates having other heteroatoms in the framework structure, such as Ga, Sn, or Zn, or silicoaluminophosphates having other heteroatoms in the framework structure. Mordenite or other solid acid catalysts can also be used as the catalyst.

In various aspects, the exposure of the olefin-containing product to the acidic catalyst can be performed in any convenient manner, such as exposing the olefin-containing product to the acidic catalyst under fluidized bed conditions, moving bed conditions, and/or in a riser reactor. In some aspects, the particle size of the catalyst can be selected in accordance with the fluidization regime which is used in the process. Particle size distribution can be important for maintaining turbulent fluid bed conditions as described in U.S. Pat. No. 4,827,069 and incorporated herein by reference. Suitable particle sizes and distributions for operation of dense fluid bed and transport bed reaction zones are described in U.S. Pat. Nos. 4,827,069 and 4,992,607 both incorporated herein by reference. Particle sizes in both cases will normally be in the range of 10 to 300 microns, typically from 20 to 100 microns.

Acidic zeolite catalysts suitable for use as described herein can be those exhibiting high hydrogen transfer activity and having a zeolite structure of ZSM-5, ZSM-11, ZSM-12, ZSM-22, ZSM-23, ZSM-35, ZSM-48, MCM-22, MCM-36, MCM-49, zeolite Y, and zeolite beta. Such catalysts can be capable of oligomerizing olefins from the olefin-containing feed. For example, such catalysts can convert C2-C4 olefins, such as those present in a refinery fuel gas, to C5+ olefins.

ZSM-5 crystalline structure is readily recognized by its X-ray diffraction pattern, which is described in U.S. Pat. No. 3,702,866. ZSM-11 is disclosed in U.S. Pat. No. 3,709,979, ZSM-12 is disclosed in U.S. Pat. No. 3,832,449, ZSM-22 is disclosed in U.S. Pat. No. 4,810,357, ZSM-23 is disclosed in U.S. Pat. Nos. 4,076,842 and 4,104,151, ZSM-35 is disclosed in U.S. Pat. No.4,016,245, ZSM-48 is disclosed in U.S. Pat. No.4,375,573 and MCM-22 is disclosed in U.S. Pat. No. 4,954,325. The U.S. Patents identified in this paragraph are incorporated herein by reference.

While suitable zeolites having a coordinated metal oxide to silica molar ratio of 20:1 to 200:1 or higher may be used, it can be advantageous to employ aluminosilicate ZSM-5 having a silica:alumina molar ratio of about 25:1 to 70:1, suitably modified. A typical zeolite catalyst component having Bronsted acid sites can comprises, consist essentially of, or consist of crystalline aluminosilicate having the structure of ZSM-5 zeolite with 5 to 95 wt. % silica, clay and/or alumina binder.

These siliceous zeolites can be employed in their acid forms, ion-exchanged or impregnated with one or more suitable metals, such as Ga, Pd, Zn, Ni, Co, Mo, P, and/or other metals of Periodic Groups III to VIII. The zeolite may include other components, generally one or more metals of group IB, IIB, IIIB, VA, VIA or VIIIA of the Periodic Table (IUPAC).

Useful hydrogenation components can include the noble metals of Group VIIIA, such as platinum, but other noble metals, such as palladium, gold, silver, rhenium or rhodium, may also be used. Base metal hydrogenation components may also be used, such as nickel, cobalt, molybdenum, tungsten, copper or zinc.

The catalyst materials may include two or more catalytic components which components may be present in admixture or combined in a unitary multifunctional solid particle.

In addition to the preferred aluminosilicates, the gallosilicate, ferrosilicate and “silicalite” materials may be employed. ZSM-5 zeolites can be useful in the process because of their regenerability, long life and stability under the extreme conditions of operation. Usually the zeolite crystals have a crystal size from about 0.01 to over 2 microns or more, such as 0.02-1 micron.

In various aspects, the catalyst particles can contain preferably about 25 wt. % to about 40 wt. % H-ZSM-5 zeolite, based on total catalyst weight, contained within a silica-alumina matrix. Typical Alpha values for the catalyst can be about 100 or less. Sulfur conversion to hydrogen sulfide can increase as the alpha value increases.

The Alpha Test is described in U.S. Pat. 3,354,078, and in the Journal of Catalysis, Vol. 4, p. 527 (1965); Vol. 6, p. 278 (1966); and Vol. 61, p. 395 (1980), each incorporated herein by reference as to that description.

In various aspects, the olefin-containing product may be exposed to the acidic catalyst by using a moving or fluid catalyst bed reactor. In such aspects, the catalyst may be regenerated, such via continuous oxidative regeneration. The extent of coke loading on the catalyst can then be continuously controlled by varying the severity and/or the frequency of regeneration. In a turbulent fluidized catalyst bed the conversion reactions are conducted in a vertical reactor column by passing hot reactant vapor upwardly through the reaction zone and/or reaction vessel at a velocity greater than dense bed transition velocity and less than transport velocity for the average catalyst particle. A continuous process is operated by withdrawing a portion of coked catalyst from the reaction zone and/or reaction vessel, oxidatively regenerating the withdrawn catalyst and returning regenerated catalyst to the reaction zone at a rate to control catalyst activity and reaction severity to affect feedstock conversion. Preferred fluid bed reactor systems are described in Avidan et al U.S. Pat. No. 4,547,616; Harandi & Owen U.S. Pat. No. 4,751,338; and in Tabak et al U.S. Pat. No. 4,579,999, incorporated herein by reference. In other aspects, other types of reactors can be used, such as fixed bed reactors, riser reactors, fluid bed reactors, and/or moving bed reactors.

In one or more aspects, effective conversion conditions for exposing the olefin-containing product to an acidic catalyst can include a temperature of about 300° F. (149° C.) to about 900° F. (482° C.), or about 350° F. (177° C.) to about 850° F. (454° C.), or about 350° F. (177° C.) to about 800° F. (427° C.), or about 350° F. (177° C.) to about 750° F. (399° C.), or about 350° F. (177° C.) to about 700° F. (371° C.), or a temperature of at least about 400° F. (204° C.), or at least about 500° F. (260° C.), or at least about 550° F. (288° C.), or at least about 600° F. (316° C.); a pressure of about 50 psig (0.34 MPag) to about 1100 psig (7.6MPag), or a pressure of about 100 psig (0.69 MPag) to about 1000 psig (6.9 MPag), or a pressure of about 100 psig (0.69 MPag) to about 200 psig (1.4 MPag), or about 150 psig (1.0 MPag) to about 975 psig (6.7 MPag), or about 200 psig (1.4 MPag) to about 950 psig (6.6 MPag), or about 250 psig (1.7 MPag) to about 900 psig (6.2 MPag), or about 300 psig (4.1 MPag) to about 850 psig (5.9 MPag), or about 300 psig (4.1 MPag) to about 800 psig (5.5

MPag), or a pressure of at least about 50 psig (0.34 MPag), or a pressure of at least about 100 psig (0.69 MPag), or a pressure of at least about 150 psig (1.0 MPag), or a pressure of at least about 200 psig (1.4 MPag), or a pressure of at least about 250 psig (1.7 MPag), or a pressure of at least about 300 psig (4.1 MPag), or a pressure of at least about 350 psig (2.4 MPag); and a total feed WHSV of about 0.05 hr−1 to about 40 hr−1, or about 0.05 to about 30 hr−1, or about 0.1 to about 20 hr−1, or about 0.1 to about 10 hr−1. Optionally, the total feed WHSV can be about 1 hr−1 to about 40 hr−1 to improve C5+ yield.

In addition to a total feed WHSV, a WHSV can also be specified for just the olefin compounds in the feed. In other words, an olefin WHSV represents a space velocity defined by just the weight of olefins in a feed relative to the weight of catalyst. In one or more aspects, the effective conversion conditions can include an olefin WHSV of at least about 0.8 hr−1, or at least about 1.0 hr−1, or at least about 2.0 hr−1, or at least about 3.0 hr−1, or at least about 4.0 hr−1, or at least about 5.0 hr−1, or at least about 8.0 hr−1, or at least about 10 hr−1, or at least about 15 hr−1. In the same or alternative aspects, the effective conversion conditions can include an olefin WHSV of about 40 hr−1 or less, or about 30 hr−1 or less, or about 20 hr−1 or less. In certain aspects, the effective conversion conditions can include an olefin WHSV of about 0.8 hr−1 to about 30 hr−1, or about 0.8 hr−1 to about 20 hr−1, or about 0.8 hr−1 to about 15 hr−1, or about 0.8 hr−1 to about 10 hr−1, or about 0.8 hr−1 to about 7 hr−1, or about 0.8 hr−1 to about 5 hr−1, or about 1.0 hr−1 to about 30 hr−1, or about 1.0 hr−1 to about 20 hr−1, or about 1.0 hr−1 to about 15 hr−1, or about 1.0 hr−1 to about 10 hr−1, or about 1.0 hr−1 to about 7 hr−1, or about 1.0 hr−1 to about 5 hr−1, or about 2.0 hr−1 to about 30 hr−1, or about 2.0 hr−1 to about 20 hr−1, or about 2.0 hr−1 to about 15 hr−1, or about 2.0 hr−1 to about 10 hr−1, or about 2.0 hr−1 to about 7 hr−1, or about 2.0 hr−1 to about 5 hr−1, about 4.0 hr−1 to about 30 hr−1, or about 4.0 hr−1 to about 20 hr−1, or about 4.0 hr−1 to about 15 hr−1, or about 4.0 hr−1 to about 10 hr−1, or about 4.0 hr−1 to about 7 hr−1. An olefin WHSV of about 1 hr−1 to about 40 hr−1 can be beneficial for increasing the C5+ yield.

In various aspects, decreasing the temperature when the olefin WHSV is increased, e.g., when the olefin WHSV is increased above 1 hr−1, may improve product yield. For example, in such aspects, temperatures of about 600° F. (316° C.) to about 800° F. (427° C.), or about 650° F. (343° C.) to about 750° F. (399° C.) may aid in increasing product yield, such as the yield of C5+ compounds, when the olefin WHSV is increased above 1 hr−1.

The olefin oligomerization reaction conditions would typically be at a higher severity than the conditions for converting dimethyl ether to olefins. For example, olefin oligomerization may be performed at higher temperature, lower WHSV and/or higher catalyst activity than that which is employed for converting dimethyl ether to olefins. The operating severity may further be adjusted to produce desire octane or aromatics content. In addition, catalytic functions promoting hydrogen transfer, such as phosphorous addition to ZSM-5, is more beneficial in the oligomerization reaction than in the conversion of dimethyl ether to olefins. In general, the reaction conditions employed in the oligomerization reactor should be those that convert C4-olefins to gasoline boiling range molecules maximizing C5+ yield.

### Oligomerized Effluent

In various aspects, exposing an olefin-containing product to the conversion conditions discussed above can produce an oligomerized olefin effluent that includes naphtha boiling range compounds. In such aspects, the gasoline boiling range compounds in the oligomerized olefin effluent can include compounds with 5 or more carbon atoms (C5+ compounds) in an amount of at least about 20 wt. %, at least about 30 wt. %, at least about 40 wt. %, at least about 50 wt. %, at least about 65 wt. %, at least about 70 wt. %, or at least about 75 wt. %. In one or more aspects, the naphtha boiling range compounds in the oligomerized effluent can include C5+ compounds in an amount of at least about 50 wt. % of the olefin-containing feed, at least about 55 wt. %, at least about 60 wt. %, at least about 65 wt. %, at least about 70 wt. %, or at least about 75 wt. %. In various aspects, the gasoline boiling range compounds in the oligomerized effluent can have an aromatic content of less than about 25 wt. %, less than about 15 wt. %, less than about 10 wt. %, or less than about 5 wt. %. In one or more aspects, the gasoline boiling range compounds in the oligomerized effluent can have a reduced sulfur content compared to the olefin-containing feed. In such aspects, the sulfur content of gasoline boiling range compounds in the oligomerized olefin effluent can be about 100 wppm or less, or about 75 wppm or less, or about 50 wppm or less, or about 30 wppm or less, or about 20 wppm or less, or about 10 wppm or less.

The oligomerized effluent may further comprise hydrocarbons lighter than the gasoline boiling range, such as C4 hydrocarbons. Such hydrocarbons may be separated from the oligomerized effluent, such as by use of a debutanizer.

### Embodiments

The following embodiments are also contemplated:

Embodiment 1—A method of converting methanol to gasoline comprising: (a) converting methanol to an intermediate stream comprising dimethyl ether; (b) exposing the dimethyl ether to a first catalyst in a reactor under first effective conversion conditions to produce an olefin-rich intermediate product, the first effective conversion conditions including an outlet temperature of less than 750° F.; and (c) exposing the olefin-rich intermediate product to a conversion catalyst under second effective conversion conditions to form an oligomerized olefin effluent comprising C5+ olefinic compounds, wherein the second effective conversion conditions comprise a pressure of less than about 500 psig and a temperature of at least about 700° F.

Embodiment 2—The method of any other enumerated Embodiment, wherein the olefin-rich intermediate product comprises at least 50 wt % olefins.

Embodiment 3—The method of claim 1, wherein the olefin-rich intermediate product comprises at least 15 wt % C1-C4 hydrocarbons.

Embodiment 4—The method of any other enumerated Embodiment, wherein the first effective conditions include an inlet temperature of less than 650° F.

Embodiment 5—The method of any other enumerated Embodiment, wherein the first catalyst comprises ZSM-5.

Embodiment 6—The method of any other enumerated Embodiment, wherein the conversion catalyst comprises ZSM-5.

Embodiment 7—The method of any other enumerated Embodiment, wherein steps (b) and (c) are performed without intermediate separation of the olefin-rich product produced in step (b).

Embodiment 8—The method of any other enumerated Embodiment, wherein the outlet temperature is less than 700° F.

Embodiment 9—The method of any other enumerated Embodiment, wherein steps (a) and (b) are performed in a first fluid bed reactor.

Embodiment 10—The method of any other enumerated Embodiment, wherein step (c) is performed in a second fluid bed reactor.

Embodiment 11—The method of any other enumerated Embodiment, wherein step (c) is also performed in the first fluid bed reactor.

Embodiment 12—The method of any other enumerated Embodiment, wherein the olefin-rich intermediate product comprises less than 30 wt % C5+ aromatics.

Embodiment 13—The method of any other enumerated Embodiment, wherein the olefin-rich intermediate product comprises less than 25 wt % C5+ aromatics.

Embodiment 14—The method of any other enumerated Embodiment, wherein the olefin-rich intermediate product comprise less than 7 wt % C10+ aromatics.

Embodiment 15—The method of any other enumerated Embodiment, wherein the olefin-rich intermediate product comprise less than 5 wt % C10+ aromatics.

1. A method of converting methanol to gasoline comprising:
(a) converting methanol to an intermediate stream comprising dimethyl ether;
(b) exposing the dimethyl ether to a first catalyst in a reactor under first effective conversion conditions to produce an olefin-rich intermediate product, the first effective conversion conditions including an outlet temperature of less than 750° F.; and
(c) exposing the olefin-rich intermediate product to a conversion catalyst under second effective conversion conditions to form an oligomerized olefin effluent comprising C₅+ olefinic compounds, wherein the second effective conversion conditions comprise a pressure of less than about 500 psig and a temperature of at least about 700° F. (371° C.).

2. The method of claim 1, wherein the olefin-rich intermediate product comprises at least 50 wt % olefins.

3. The method of claim 1, wherein the olefin-rich intermediate product comprises at least 15 wt % C1-C4 hydrocarbons.

4. The method of claim 1, wherein the first effective conditions include an inlet temperature of less than 650° F.

5. The method of claim 1, wherein the first catalyst comprises ZSM-5.

6. The method of claim 1, wherein the conversion catalyst comprises ZSM-5.

7. The method of claim 1, wherein steps (b) and (c) are performed without intermediate separation of the olefin-rich product produced in step (b).

8. The method of claim 1, wherein the outlet temperature is less than 700° F.

9. The method of claim 1, wherein steps (a) and (b) are performed in a first fluid bed reactor.

10. The method of claim 8, wherein step (c) is performed in a second fluid bed reactor.

11. The method of claim 9, wherein step (c) is also performed in the first fluid bed reactor.

12. The method of claim 1, wherein the olefin-rich intermediate product comprises less than 30 wt % C5+ aromatics.

13. The method of claim 12, wherein the olefin-rich intermediate product comprises less than 25 wt % C5+ aromatics.

14. The method of claim 1, wherein the olefin-rich intermediate product comprise less than 7 wt % C10+ aromatics.

15. The method of claim 14, wherein the olefin-rich intermediate product comprise less than 5 wt % C10+ aromatics.
